(19) United States
(12) Patent Application Publication
(10) Pub. No.: US 2013/0296602 A1
(43) Pub. Date: Nov. 7, 2013

(54) Conversion of Methyl-2-Acetoxy Propionate to Methyl Acrylate and Acrylic Acid

(71) Applicants: Jane Ellen Godlewski, Loveland, OH (US); Juan Esteban Velásquez, Cincinnati, OH (US); Dimitris Ioannis Collias, Mason, OH (US)

(72) Inventors: Jane Ellen Godlewski, Loveland, OH (US); Juan Esteban Velásquez, Cincinnati, OH (US); Dimitris Ioannis Collias, Mason, OH (US)

(21) Appl. No.: 13/465,991

(22) Filed: May 7, 2012

(51) Int. Cl. C07C 51/09; C07C 67/333

(52) U.S. Cl. 560/211; 562/598

(57) ABSTRACT

Disclosed herein is a method that includes contacting (a) a gaseous mixture that includes (i) an inert gas and (ii) a vaporized liquid feed containing methyl-2-acetoxy propionate (MAPA) and an excipient, with (b) a material having a surface acidity of about 5 micromoles per gram (μmol/g) or less and a surface basicity of about 15 μmol/g or less, under conditions sufficient to produce a conversion product that includes methyl acrylate in a molar yield of at least about 85% from MAPA. The excipient is selected from the group consisting of acetic acid, formic acid, methyl acetate, lactic acid, carbon dioxide, and mixtures thereof. This method now makes possible the ability to further process the manufactured methyl acrylate into acrylic acid by either of two general routes that ultimately produce acrylic acid in a molar yield of at least about 80% from methyl acrylate and, preferably, substantially free of propanoic acid.

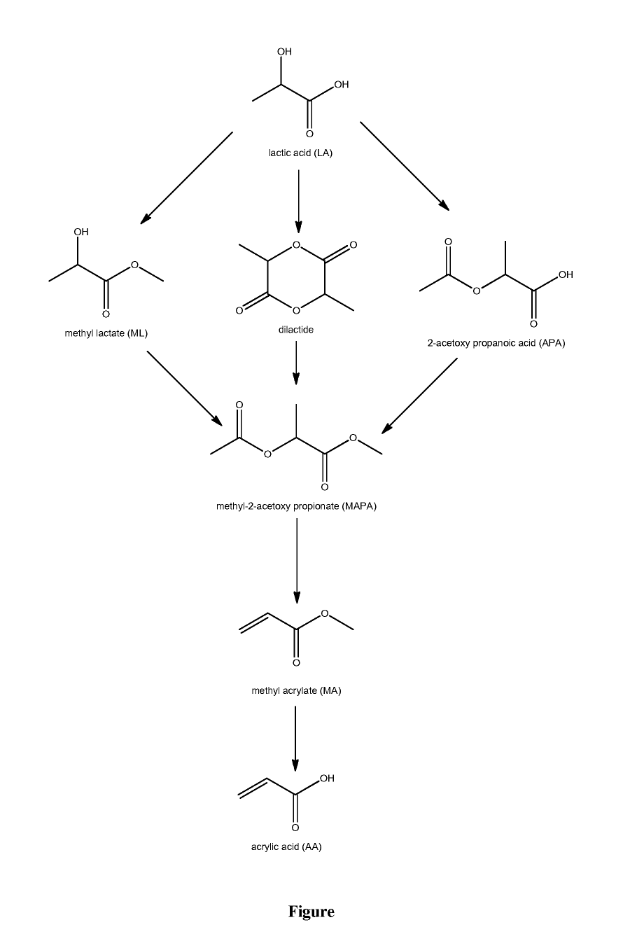

### BACKGROUND OF THE INVENTION

1. Field of the Disclosure

The disclosure generally relates to the conversion of methyl-2-acetoxy propionate (“MAPA”) to methyl acrylate which, in turn, can be converted more conveniently into acrylic acid, without significant conversion of MAPA to impurities, such as methyl propionate.

2. Brief Description of Related Technology

Acrylic acid has a variety of industrial uses, typically consumed in the form of a polymer. In turn, these polymers are commonly used in the manufacture of, among other things, adhesives, binders, coatings, paints, polishes, and superabsorbent polymers, which are used in disposable absorbent articles including diapers and hygienic products, for example. Acrylic acid is commonly made from petroleum sources. For example, acrylic acid has long been prepared by catalytic oxidation of propylene. These and other methods of making acrylic acid from petroleum sources are described in Kirk-Othmer Encyclopedia of Chemical Technology, Vol. 1, pgs. 342-69 (5^{th} Ed., John Wiley & Sons, Inc., 2004).

Increasingly, however, there is interest in making acrylic acid from non-petroleum based sources, such as lactic acid. U.S. Pat. Nos. 4,729,978 and 4,786,756 generally describe the conversion of lactic acid to acrylic acid. The conversion described in these patents is a direct conversion. Lactic acid, however, also can be converted indirectly to acrylic acid. Specifically, the sole FIGURE illustrates three routes in the manufacture of acrylic acid from lactic acid. These routes include the intermediate products methyl-2-acetoxy propionate (hereinafter MAPA) and methyl acrylate.

The art has long focused on converting MAPA to methyl acrylate. For example, Burns et al. (1935) J. Chem. Soc. 400-06, described the decomposition (via pyrolysis) at 500° C. of one mole of MAPA to produce one mole of methyl acrylate and one mole of acetic acid. Shortly thereafter, Smith et al. (1942) Ind. Eng. Chem. 34:473-79, taught the same decomposition but were able to obtain conversion of almost all of the MAPA at temperatures in excess of 550° C. in the presence of several different contact materials (e.g., quartz having mesh size 8). Further research by Fisher et al. (1944) Ind. Eng. Chem. 36:229-34, led the art to conclude that no particular material (active catalyst or inert packing) appreciably favors decomposition of MAPA to high yields of methyl acrylate. And, while all of these investigators reported high molar selectivities of methyl acrylate from MAPA, low conversions of MAPA were achieved. Thus, for example, the processes Fisher et al. described generally required relatively low gas hourly space velocities (i.e., relatively high residence times) and often multiple passes of the reactants over the packing material (whether catalytic or otherwise). Subsequent developments in the art suggest that methyl acrylate selectivity decreases when the process is operated at elevated pressure, despite the concomitant increased throughput resulting from the higher pressure.

Ultimately, both high conversion of MAPA and high selectivity of methyl acrylate are desired. And, with that, of course, a low yield of by-products is preferred, especially when the obtained methyl acrylate can be expected to undergo further processing to produce acrylic acid. A highly pure acrylic acid free of, for example, acetic acid, acetaldehyde, and propanoic acid, is often the only variety that is acceptable in the manufacture of super absorbent polymers used, for example, in absorbent articles expected to contact human skin, such as diapers and feminine hygiene products. In the field of those products, it is generally known that aldehydes prohibit polymerization and that acetic acid and propanoic acid impart the superabsorbent polymer with an undesirable odor. See generally, US 2011/0306732.

Even when impurities are present among the highly pure acrylic acid in only small amounts, these impurities impose additional costs in processing acrylic acid in the manufacture of superabsorbent polymers, for example. And the literature regarding the manufacture of these polymers is replete with potential solutions—expensive as they may be—to removing impurities (like acetic acid and propanoic acid) when present among the manufactured acrylic acid in merely small amounts. For example, U.S. Pat. No. 6,541,665 B1 describes the purification of acrylic acid containing propanoic acid, furans, water, acetic acid and aldehydes by crystallization, distillation, and recycling. The '665 patent reports that a 5-stage crystallization (two purification stages and three stripping stages) was effective to obtain 99.94% acrylic acid from a 99.48% acrylic acid mixture containing 2600 parts per million (weight basis) (ppm) acetic acid and 358 ppm propanoic acid, among others. Similarly, U.S. patent application Publication No. 2011/0257355 describes a method of removing propanoic acid in a single pass crystallization from a crude reaction mixture (containing acrylic acid) derived from glycerol dehydration/oxidation to obtain 99% acrylic acid. These purification methods are necessary to obtain a highly pure acrylic acid necessary for downstream uses in, for example, the manufacture of superabsorbent polymers. Thus, there is certainly value in eliminating impurities, if at all possible, if only to be able to employ these purification methods. And eliminating impurities in the reaction routes discussed above, certainly includes eliminating impurities in the product of the conversion of MAPA to methyl acrylate.

But, heretofore, the manufacture of acrylic acid from lactic acid by the chemical routes depicted in the sole FIGURE, and described in the literature cited above, leads to significant amounts of undesired by-products—indeed amounts of by-products far too high to even utilize the purification methods identified in the preceding paragraph. Of course, the low selectivity for acrylic acid in these processes also leads to a loss of feedstock, and ultimately leads to increased production costs. Thus, the art does not provide or teach a commercially viable process for converting MAPA to methyl acrylate and eventually to acrylic acid.

### SUMMARY OF THE INVENTION

It has now been found that methyl acrylate can be manufactured from methyl-2-acetoxy propionate (MAPA) under certain conditions such that methyl acrylate is produced in a molar yield of at least about 85% from MAPA. Further, these conditions are also sufficient to result in a molar yield of methyl propionate of about 5% or less, and highly preferably substantially free of methyl propionate. This is a marked advance in the art because it is now possible to more efficiently manufacture highly pure acrylic acid indirectly from MAPA.

Accordingly, in one embodiment of a method of making methyl acrylate, the method includes contacting (a) a gaseous mixture that includes (i) an inert gas and (ii) a vaporized liquid feed containing MAPA and an excipient, with (b) a material having a surface acidity of about 5 micromoles per gram (μmol/g) or less and a surface basicity of about 15 mmol/g or less, under conditions sufficient to produce a conversion product that includes methyl acrylate in a molar yield of at least about 85% from MAPA. The excipient is selected from the group consisting of acetic acid, formic acid, methyl acetate, lactic acid, carbon dioxide, and mixtures thereof.

In one particularly preferred embodiment, the method includes contacting (a) a gaseous mixture that includes (i) nitrogen and (ii) a vaporized liquid feed containing MAPA and acetic acid, with (b) a fused quartz at a temperature of about 560° C. at a gas hourly space velocity (GHSV) of about 450 per hour (h⁻¹) to produce a conversion product containing methyl acrylate in a molar yield of at least about 90% from MAPA, wherein MAPA is present in the gaseous mixture in a concentration of about 20 mol. %, based on the total moles of the gaseous mixture, and acetic acid is present in the liquid feed in a concentration of about 20 wt. %, based on the total weight of MAPA and excipient in the liquid feed. In another particularly preferred embodiment, the method includes contacting (a) a gaseous mixture that includes (i) nitrogen and (ii) a vaporized liquid feed containing MAPA and acetic acid, with (b) a fused quartz at a temperature of about 560° C. at a GHSV of about 225 h⁻¹ to produce a conversion product that includes methyl acrylate in a molar yield of at least about 90% from MAPA, wherein MAPA is present in the gaseous mixture in a concentration of about 30 mol. %, based on the total moles of the gaseous mixture, and acetic acid is present in the liquid feed in a concentration of about 40 wt. %, based on the total weight of MAPA and excipient in the liquid feed.

These embodiments make possible the ability to further process the manufactured methyl acrylate into acrylic acid by one of a two general routes. In one embodiment, therefore, the method further includes contacting the conversion product containing methyl acrylate in a vaporized mixture that includes at least one of water and an organic carboxylic acid with a catalyst under conditions sufficient to produce a reaction product containing acrylic acid in a molar yield of at least about 80% from methyl acrylate. In a more preferred embodiment, the reaction product includes a molar yield of about 5% or less of propanoic acid from methyl acrylate, and in an even more preferred embodiment, the reaction product is substantially free of propanoic acid.

In alternative embodiments, and according to a second general route for the conversion of MAPA to methyl acrylate, the method includes combining the conversion product containing methyl acrylate with an aqueous solution of a base under conditions sufficient to produce a reaction product that includes a salt of acrylic acid in a molar yield of at least about 80% from methyl acrylate. Preferred embodiments may further include removing (by distillation) methanol present in the reaction product, thereby providing a more pure salt of acrylic acid. In a further preferred embodiment, the method may include mixing the salt of the acrylic acid with a strong mineral acid to produce acrylic acid. The result of these two general routes is an acrylic acid product sufficient for conventional industrial uses and one that may not require the complicated purification presently required in the art.

Additional features of the invention may become apparent to those skilled in the art from a review of the following detailed description, taken in conjunction with the drawing, the examples, and the appended claims.

### BRIEF DESCRIPTION OF THE DRAWING FIGURE

For a more complete understanding of the disclosure, reference should be made to the following detailed description and accompanying drawing FIGURE, which illustrates three chemical routes in the manufacture of acrylic acid from lactic acid. While the disclosed methods are susceptible of embodiments in various forms, hereinafter are described specific embodiments of the invention, with the understanding that the disclosure is intended to be illustrative, and is not intended to limit the invention to the specific embodiments described herein.

### DETAILED DESCRIPTION OF THE INVENTION

As noted above, it has now been found that methyl acrylate can be manufactured from methyl-2-acetoxy propionate (MAPA) under certain conditions such that methyl acrylate is produced in a molar yield of at least about 85% from MAPA. Further, these conditions are also sufficient to result in a molar yield of methyl propionate of about 5% or less, and highly preferably substantially free of methyl propionate. This is a marked advance in the art because it is now possible to more efficiently manufacture highly pure acrylic acid indirectly from MAPA by at least one of two general chemical routes. Ultimately, the result of the discovery presented herein is that an acrylic acid product can be manufactured from MAPA that is sufficient for conventional industrial uses and one that may not require the complicated purification presently required in the art.

Generally, the method of making methyl acrylate includes contacting (a) a gaseous mixture that includes (i) an inert gas and (ii) a vaporized liquid feed containing MAPA and an excipient, with (b) a material having a surface acidity of about 5 micromoles per gram (μmol/g) or less and a surface basicity of about 15 mmol/g or less, under conditions sufficient to produce a conversion product that includes methyl acrylate in a molar yield of at least about 85% from MAPA. The excipient is selected from the group consisting of acetic acid, formic acid, methyl acetate, lactic acid, carbon dioxide, and mixtures thereof. Preferably, the excipient is acetic acid. In case that the excipient is gas under ambient conditions (e.g. carbon dioxide), then the excipient can be the inert gas or part of it, or can be blended with the vaporized liquid feed.

Preferably, the gas hourly space velocity (GHSV) is about 225 per hour (h⁻¹) to about 900 h⁻¹, more preferably about 225 h⁻¹ to about 600 h⁻¹. Under these preferred gas hourly space velocities, preferably the excipient is present in the liquid feed in a concentration of about 20 wt. % to about 40 wt. %, based on the total weight of MAPA and excipient in the liquid feed, and MAPA is present in the gaseous mixture in a concentration of about 20 mol. % to about 30 mol. %, based on the total moles of the gaseous mixture. Alternatively under these preferred gas hourly space velocities, the excipient preferably is present in the liquid feed in a concentration of about 2 wt. % to about 20 wt. %, based on the total weight of MAPA and excipient in the liquid feed, and MAPA is present in the gaseous mixture in a concentration of about 5 mol. % to about 20 mol. %, based on the total moles of the gaseous mixture. The above ranges of excipient concentration in the liquid feed refer to acetic acid as the excipient, in which case the range of about 2 wt. % to about 20 wt. % corresponds to about 5 mol. % to about 38 mol. %, and the range of about 20 wt. % to about 40 wt. % corresponds to about 38 mol. % to about 62 mol. %. If the excipient is other than acetic acid, then the preferred ranges of excipient concentration in the liquid feed are about 5 mol. % to about 38 mol. % and about 38 mol. % to about 62 mol. %.

Not wishing to be bound by theory, the presence of the excipient (e.g. acetic acid, formic acid, methyl acetate, lactic acid, carbon dioxide, and mixtures thereof) in the range from 2 wt. % to 40 wt. % in the MAPA liquid feed is important to achieving the unexpectedly high yields of methyl acrylate from MAPA due to the effect that the excipient has on the (packing) material. More specifically, it is believed that the excipient interacts with active sites of the material and, thus, it reduces the probability of side reactions taking placing. Excipient concentrations less than 2 wt. % causes incomplete interaction with the active sites thus allowing for some side reactions to take place and reducing the yield of methyl acrylate from MAPA. On the other hand, excipient concentrations greater than 40 wt. % may cause the formation of decarboxylation products from the excess excipient (i.e., amount in excess of the amount that causes the full interaction with the active sites of the material) that can then react with methyl acrylate, thus, reducing its yield from MAPA. The gaseous mixture will include MAPA, of course. But, it has been discovered that the conversion of MAPA to methyl acrylate, methyl acrylate yield, and accordingly selectivity for methyl acrylate all improve—and quite unexpectedly so—when the gaseous mixture includes the excipient in the indicated amount.

In addition to MAPA and the excipient, the gaseous mixture also includes an inert gas. Preferably, the inert gas is substantially free, if not completely free, of oxygen. In this context, the term “substantially free” refers to a molar concentration of less than about 1 mol. %, based on the total moles of the mixture. Preferably, the inert gas is selected from the group consisting of nitrogen, helium, neon, argon, carbon monoxide, carbon dioxide, and mixtures thereof. More preferably, the inert gas is at least one of nitrogen and carbon dioxide.

The source of MAPA does not affect the method disclosed and claimed herein. MAPA can be readily purified by methods well known by those ordinarily-skilled in the art. MAPA can be introduced into the method of this invention in a form that may include impurities attendant with its manufacture or in a purified form (essentially free of such impurities). If present, the impurities may be expected to include lactic acid, methanol, methyl acetate, methyl lactate, acetoxy propanoic acid, lactide (e.g., dilactide), acetic acid, acetic anhydride, sulfuric acid, and mixtures thereof. If present, preferably, the impurities are only about 10 weight percent (wt. %) or less of the gaseous mixture (at standard temperature and pressure), more preferably, about 5 wt. % or less, and even more preferably about 2 wt. % or less.

There is a variety of conditions sufficient to convert the MAPA present in the gaseous mixture to a conversion product that includes methyl acrylate in a molar yield of at least about 85% from MAPA. For example, one such condition is that the method is carried out in a reactor. The surfaces of the reactor likely to come into contact with the gaseous mixture or conversion product (i.e., the contacting surfaces) preferably are constructed of quartz or stainless steel. Thus, for example, the conditions can include a stainless steel reactor, the interior surfaces (or contacting surfaces) of which are quartz lined. Preferably, the reactor has an aspect ratio (length/diameter) of at least about 6, and more preferably about 6 to about 24.

The material packing the reactor and which the gaseous mixture contacts to produce the conversion product preferably has a surface acidity of about 5 micromoles per gram (μmol/g) or less. The material also preferably has a surface basicity of about 15 μmol/g or less.

Exemplary materials suitable for use in accordance with the method include those selected from the group consisting of silicates, aluminates, carbons, titanium oxides, and mixtures of the same. If a silicate is used, then preferably it is selected from the group consisting of quartz, fused quartz, or mixtures thereof. As exemplified below, more highly preferred is quartz and, specifically a fused quartz having mesh size of 4 to 50, more preferably, a mesh size of 35 to 50. If the material is a carbon, then preferably it is one selected from the group consisting of graphite, graphene, diamond, and mixtures thereof. Among this group, more highly preferred are diamonds, such as those having a mesh size of 60 to 80. Synthetic diamonds having such mesh sizes may be used according to the inventive method.

Among other conditions sufficient to achieve the conversion and yields noted above, are temperature, pressure, and gas hourly space velocity (GHSV) of the gaseous mixture. For example, the temperature at which the gaseous mixture contacts the packing material preferably is about 500° C. to about 580° C., more preferably about 540° C. to about 575° C., and even more preferably about 550° C. to about 565° C. The lower temperatures within this range may lead to a conversion product that contains a lower molar yield of methyl acrylate due to lower conversion of MAPA to methyl acrylate. The higher temperatures within this range may result in lower molar yield of methyl acrylate, because of decarboxylation/decarbonylation of the produced methyl acrylate. Accordingly, it is believed that a temperature of about 550° C. to about 565° C. generally should provide the maximum conversion of MAPA to methyl acrylate and also the greatest molar yield of methyl acrylate from MAPA under the other specified conditions.

Preferably, the gaseous mixture contacts the packing material at a gas hourly space velocity (GHSV) of about 225 per hour (h⁻) to about 900 h⁻¹, more preferably about 225 h⁻¹ to about 600 h⁻¹, and even more preferably about 450 h⁻¹. GHSV values in the higher end of the broad range (i.e., shorter residence times) are likely to result in a conversion product containing lower amounts of by-products of the conversion of MAPA, such as methyl propionate, and perhaps also a greater amount of MAPA that has not undergone the conversion to methyl acrylate; though, amounts of MAPA still manageable for downstream use of the conversion product. GHSV values in the lower end of the broad range (i.e., longer residence times) are likely to result in a conversion product containing greater amounts of by-products of the conversion of MAPA, and perhaps a smaller amount of MAPA that has not undergone the conversion to methyl acrylate; though, again, an amount of by-products still manageable for downstream use of the conversion product. Based in part on the data reported below, it is believed that a GHSV of about 225 h⁻¹ to about 600 h⁻¹ is likely to provide the maximum conversion of MAPA to methyl acrylate and also the greatest molar yield of methyl acrylate from MAPA under the other specified conditions.

Preferably, the pressure is maintained at zero pounds per square inch gauge (psig) to about 100 psig, more preferably at zero psig (atmospheric pressure). This preference is based on a determination that pressures in excess of 100 psig lead to reduced yields of methyl acrylate from MAPA and also undesired by-products.

As noted above, the general method of making methyl acrylate from MAPA results in a conversion product that includes methyl acrylate in a molar yield of at least about 85% from MAPA. The conversion product is merely the output from the reactor in which the MAPA is contained. Thus, the conversion product, as recited herein, would include any unreacted feed components, such as acetic acid, methyl acetate (if present), MAPA, and impurities (if present). Conversion, yield, and selectivity are defined as follows:

Methyl acrylate(MA)Molar Yield=(moles of MA product out÷moles of MAPA feed in)×100.

MAPA Molar Conversion=(1−(moles of MAPA feed out÷moles of MAPA feed in))×100.

MA Molar Selectivity=(MA Molar Yield÷MAPA Molar Conversion)×100.

In successively preferred embodiments, the molar yield of methyl acrylate from MAPA is at least about 90%, and at least about 95%.

Among the many benefits, is the low amount of by-products that result from the MAPA conversion specified. Specifically, the conversion product highly preferably is substantially free, if not completely free, of methyl propionate. In embodiments, where, however, some methyl propionate may be present in the conversion product that contains the methyl acrylate, the molar yield of methyl propionate from MAPA is only about 5% or less, preferably about 4% or less, more preferably about 3% or less, even more preferably about 2% or less, and still more preferably about 1% or less. In the context of methyl propionate and the conversion product, the term “substantially free” refers to instances where methyl propionate is either not detectable or is present in a detectable amount of a molar yield of about 0.5% or less.

As noted above, the ability now to manufacture, through high conversion of MAPA, highly pure methyl acrylate with low levels of impurities, and a high molar yield of methyl acrylate from MAPA, marks an important advance. This advance is further demonstrated by a number of examples set forth below. As a result of this advance, it is now possible to more efficiently manufacture highly pure acrylic acid indirectly from MAPA by at least one of two general chemical routes that will now be described in further detail. Ultimately, the result of the discovery presented herein is that an acrylic acid product can be manufactured from MAPA that is sufficient for conventional industrial uses and one that may not require the complicated purification presently required in the art.

The two general chemical routes for making acrylic acid from the methyl acrylate obtained in the conversion product from the MAPA reactor do not require purification of the methyl acrylate, but can of course lead to improved results if such purification is undertaken. Purification methods include distillation and extraction, which are methods, in this context generally known by ordinarily-skilled artisans.

According to one of these general chemical routes, the method discussed above further includes contacting the conversion product in a vaporized mixture that includes at least one of water and an organic carboxylic acid with a catalyst under conditions sufficient to produce a reaction product containing acrylic acid in a molar yield of at least about 80% from methyl acrylate. The vaporized mixture here can include water, an organic carboxylic acid, or both. As previously noted, the conversion product can include methyl acetate, or it can be free of methyl acetate. The presence of methyl acetate in the conversion product may require adjustments in the amount of organic carboxylic acid used in instances where the acid is acetic acid and water is not part of the vaporized mixture. For example, when methyl acetate is present in the conversion product, but no water is present in the vaporized mixture, the amount of acetic acid (if that is the organic carboxylic acid) likely needs to be increased relative to the amount of acid that would otherwise be employed where the conversion product lacks methyl acetate and the vaporized mixture lacks water.

The vaporized mixture here can include water, an organic carboxylic acid, or both. Regardless of which of these permutations is present in any particular embodiment, preferably the vaporized mixture includes a molar ratio of (a) methyl acrylate to (b) at least one of water and organic carboxylic acid of about 1:80 to about 1:200.

When present, preferably the organic carboxylic acid is selected from the group consisting of formic acid, acetic acid, butyric acid, isobutyric acid, pentanoic acid, 3-methylbutanoic acid, and mixtures thereof. In further preferred embodiments, the organic carboxylic acid is either formic acid or acetic acid.

The vaporized mixture can include an inert gas. Preferably, the inert gas is selected from the group consisting of nitrogen, argon, helium, carbon monoxide, carbon dioxide, and mixtures thereof. More preferably, the inert gas is nitrogen.

The catalyst preferably has a surface area of at least about 1 m²/g, preferably at least about 100 m²/g. Generally, the higher surface area the greater potential for a reaction product having a high acrylic acid content. The catalyst preferably has a surface acidity of at least about 300 micromoles per gram (μmol/g).

The catalyst is selected from the group consisting of alumina, silica, silicates, aluminosilicates, silicoaluminophosphates, aluminophosphates, metal oxides, Group I and II sulfates, phosphates, pyrophosphates, polyphosphates, polysulfonate salts, and mixtures thereof. Preferably, the catalyst is selected from the group consisting of alumina, aluminosilicates, silicoaluminophosphates, and aluminophosphates. Highly preferably, the catalyst includes γ-alumina. The catalyst can include an inert support that preferably is constructed of a material selected from the group consisting of carbon, silicates, aluminates, and combinations thereof. Generally a catalyst that includes an inert support can be made by one of two exemplary methods: impregnation or co-precipitation. In impregnation, a suspension of the solid inert support is treated with a solution of a pre-catalyst, and the resulting material is then activated under conditions that will convert the pre-catalyst to a more active state. In co-precipitation, a homogenous solution of the catalyst ingredients is precipitated by the addition of additional ingredients.

The reaction can be carried out under a variety of conditions sufficient to produce a reaction product containing acrylic acid in a molar yield of at least about 80% from methyl acrylate. For example, one such condition is that the reaction is carried out in a reactor. The surfaces of the reactor likely to come into contact with the conversion product, vaporized mixture, or reaction product (i.e., the contacting surfaces) preferably are constructed of quartz or stainless steel. Thus, for example, the conditions can include a stainless steel reactor, the interior surfaces (or contacting surfaces) of which are quartz lined. The material of construction may activate side reactions. For example, transition metals, if present on the contact surfaces of the reactor, may favor reduction of acrylic acid to propanoic acid when molecular hydrogen is present. As it is important to avoid these types of reactions if possible, it is important to select a reactor with contact surfaces that will not promote such reactions. Preferably, the reactor has an aspect ratio (length/diameter) of at least about 6, and more preferably about 6 to about 24.

Among other conditions sufficient to produce a reaction product containing acrylic acid in a molar yield of at least about 80% from methyl acrylate noted above, are temperature, pressure, and gas hourly space velocity of the vaporized mixture. For example, the temperature at which the vaporized mixture contacts the catalyst preferably is about 150° C. to about 500° C., more preferably about 250° C. to about 300° C. when the catalyst includes γ-alumina. The higher temperatures within this range may lead to a reaction product that contains a lower molar yield of acrylic acid and potentially higher amounts of undesired by-products, though amounts of such by-products that are likely still manageable for downstream purification of the reaction product to obtain a highly pure acrylic acid.

Preferably, the vaporized mixture contacts the catalyst at a GHSV of about 180 per hour (h⁻¹) to about 1800 h⁻¹, more preferably about 240 h⁻¹ to about 720 h⁻¹, and even more preferably about 350 h⁻¹. GHSV values in the higher end of the broad range (i.e., shorter residence times) are likely to result in a reaction product containing higher amounts of methyl acrylate. GHSV values in the lower end of the broad range (i.e., longer residence times) are likely to result in a conversion product containing higher amounts of by-products of the reaction of methyl acrylate, and perhaps a lower amount of methyl acrylate that has not undergone the conversion to acrylic acid, though, again, amounts of each still manageable for downstream use of the reaction product.

Preferably, the pressure is maintained at zero psig to about 100 psig, more preferably at zero psig (atmospheric pressure). While not wishing to be bound to any particular theory, it is believed that higher pressures within this range leads to increased reaction rates and conversion (as a result of high reagent concentration), but lower selectivity for acrylic acid. This effect may be modulated by changing the residence time.

Among the many benefits of this aspect of the method, is the low amount of by-products that result from the catalytic reaction of methyl acrylate. Specifically, the reaction product highly preferably is substantially free, if not completely free, of propanoic acid. In embodiments, where, however, some propanoic acid may be present in the reaction product that contains the acrylic acid, the molar yield of propanoic acid from methyl acrylate is only about 5% or less, preferably about 4% or less, more preferably about 3% or less, even more preferably about 2% or less, and still more preferably about 1% or less. In the context of propanoic acid and the reaction product, the term “substantially free” refers to an instance where propanoic acid is either not detectable or is present in a detectable amount of a molar yield of about 0.1% or less.

According to the other of the two general chemical routes to obtaining acrylic acid from methyl acrylate, the method of converting MAPA to methyl acrylate discussed above further includes combining the conversion product with an aqueous solution of a base under conditions sufficient to produce a reaction product containing a salt of acrylic acid in a molar yield of at least about 80% from methyl acrylate.

Preferably the base is selected from at least one of hydroxides, carbonates, and bicarbonates of: ammonium, Group I metals, or Group II metals. Thus, the base, can be a Group I hydroxide, a Group II hydroxide, an ammonium hydroxide, a Group I carbonate, a Group II carbonate, an ammonium carbonate, a Group I bicarbonate, a Group II bicarbonate, an ammonium bicarbonate, or mixtures thereof. Preferably, however, the base is an alkali hydroxide selected from the group consisting of lithium hydroxide, sodium hydroxide, potassium hydroxide, rubidium hydroxide, cesium hydroxide, and mixtures thereof. More highly preferred is a sodium hydroxide or potassium hydroxide.

Methanol is an expected part of this reaction product. Accordingly, the method can include removing methanol from the reaction product by distillation. Thereafter, the method may further include mixing the salt of the acrylic acid with a strong mineral acid to produce acrylic acid. Preferably, the strong mineral acid is selected from the group consisting of hydrochloric acid, sulfuric acid, phosphoric acid, and mixtures thereof. The conditions sufficient to produce the reaction product in this scheme include a temperature of 0° C. to about 100° C., preferably about 5° C. to about 50° C., and more preferably at room temperature.

### EXAMPLES

The following examples are provided to illustrate the invention, but are not intended to limit the scope thereof.

Equipment Set-Up and Design

A ½ inch 316 stainless steel tube was packed with 10 g of fused quartz that was ground and sieved to 300-500 μm to give 3.5 inch bed depth. The tube was set up in a down-flow arrangement and was equipped with a Knauer Smartline 100 feed pump (Knauer GmbH; Berlin, Germany), a Brooks 0254 gas flow controller (Brooks Instrument Inc.; Hatfield, Pa.), a Brooks back pressure regulator and a catch tank. All connections were made using 316 stainless steel Swagelok fittings. The steel tube was placed inside an aluminum block that was heated using an Applied Test Systems clam shell furnace series 3210 (Applied Test Systems Inc.; Butler, Pa.) to give an 8 inch heated zone. The reactor jacket was preheated to 550° C. under nitrogen. The liquid conversion product was captured in the catch tank for analysis by off-line high performance liquid chromatography (HPLC) using an Agilent 1100 HPLC (Agilent Technologies Inc.; Santa Clara, Calif.) equipped with a DAD detector and a Waters Atlantis T3 column (Catalog #186003748; Waters Corp.; Milford, Mass.) using methods generally known by those having ordinary skill in the art. The gaseous stream was analyzed on-line by gas chromatography (GC) using an Agilent 7890 system equipped with a FID detector and Agilient Varian CP-Para Bond Q column (Catalog #CP7351). The methyl propionate content was determined in the liquid sample by off-line GC analysis using an Agilent 7890 system equipped with a Restek Rxi®-624Sil MS column (Catalog #13868; Restek Corp.; Bellefonte, Pa.).

### DEFINITIONS

“AA” refers to acrylic acid, “MA” refers to methyl acrylate, and “MAPA” refers to methyl-2-acetoxypropionate.

AA Molar Yield=(moles of AA product out÷moles of MA feed in)×100.

MA Molar Conversion=(1−(moles of MA feed out÷moles of MA feed in))×100.

AA Molar Selectivity=(AA Molar Yield÷MA Molar Conversion)×100.

As used herein, the term “gas hourly space velocity” (GHSV) is calculated as the flow rate of the gaseous mixture under Standard Temperature and Pressure (STP; 25° C. and 0.986 atm) conditions divided by the volume of the material and is reported in units of h⁻¹.

The fused quartz used in Examples 2 through 7 was purchased from Sigma Aldrich (Catalog #342831; St. Louis, Mo.) and was ground and sieved to give particle size distribution of 300-500 μm. The surface acidity was measured using Micromeritics Autochem II 2920 (Norcross, Ga.) Thermal Programmed Desorption (TPD) equipment with ammonia gas to give 0.8 μmol/g and the surface basicity was measured using Micromeritics Autochem II 2920 TPD equipment using CO₂ gas to give 0.4 μmol/g. For the TPD experiments, the samples were pretreated at 550° C. for 30 minutes under helium (He) atmosphere. Carbon dioxide (CO₂) adsorption was carried out at 45° C. for 30 minutes and desorption was performed at 10° C. per minute up to 700° C. and hold for 30 minutes Ammonia (NH₃) adsorption was carried out at 120° C. for 30 minutes and desorption was performed at 10° C. per minute up to 700° C. and hold for 30 minutes.

The diamond used in Example 8 was purchased from Eastwind Diamond Abrasives (Catalog #E-PPM6006080; Windsor, Vt.). The surface acidity was measured using Micromeritics Autochem II 2920 Thermal Programmed Desorption (TPD) equipment with ammonia gas to give 4.0 μmol/g and the surface basicity was measured in the same equipment using CO₂ gas to give 7.7 μmol/g.

### Example 1

This Example demonstrates one way to make MAPA, and purifying the same. To methyl-L-lactate (Sigma Aldrich Co. LLC, catalog #230340, 813.2 g, 7.81 mol) was added sulfuric acid (0.8 mL). To this solution was added acetic anhydride (877 g, 8.59 mol) drop-wise at 20-40° C. with water bath cooling. The reactants were stirred until the reaction was complete as determined by GC. The resulting mixture was quenched with water (115 mL) and distilled over a 10″ Oldershaw column at 100 mbar and 97° C. to give the product as a colorless liquid (510.1 g, 44.7% yield). The product was redistilled over a12″ vigreux column at 25 mbar and 62° C. to give MAPA product (445.6 g). The obtained MAPA was used in the Examples 2 through 10.

### Example 2

Nitrogen (28 mL/min) and a liquid feed consisting of 2 wt. % acetic acid in MAPA (0.018 mL/min) were flowed over 10 g fused quartz (silicon oxide ground and sieved to 300-500 μm) at 560±10° C. and atmospheric pressure to give a GHSV of 225 h⁻¹ and a MAPA gas concentration of 10 mol. %. The reaction was equilibrated for 1 hour under flow then sampled every hour for 3 hours. The total methyl acrylate was determined by GC and HPLC analyses of three 1 hour pulls and results were averaged to give the conversion product which contained methyl acrylate in 98.7% molar conversion, 91.5% molar yield, and 92.7% molar selectivity; and methyl propionate in a molar yield of 0.4%.

### Example 3

Nitrogen (37 mL/min) and a liquid feed consisting of 20 wt. % acetic acid in MAPA (0.083 mL/min) were flowed over 10 g fused quartz (silicon oxide ground and sieved to 300-500 μm) at 560±10° C. and atmospheric pressure to give a GHSV of 400 h⁻¹ and a MAPA gas concentration of 20 mol. %. The reaction was equilibrated for 1 hour under flow then sampled every hour for 3 hours. The total methyl acrylate was determined by GC and HPLC analyses of three 1 hour pulls and results were averaged to give the conversion product which contained methyl acrylate in 98.5% molar conversion, 98.7% molar yield, and 100% molar selectivity. No methyl propionate was detected.

### Example 4

### Comparative

Nitrogen (196 mL/min) and a liquid feed consisting of 2 wt. % acetic acid in MAPA (0.13 mL/min) were flowed over 10 g fused quartz (silicon oxide ground and sieved to 300-500 μm) at 560±10° C. and atmospheric pressure to give a GHSV of 1800 h⁻¹ and a MAPA gas concentration of 10 mol. %. The reaction was equilibrated for 1 hour under flow then sampled every hour for 3 hours. The total methyl acrylate was determined by GC and HPLC analyses of three 1 hour pulls and results were averaged to give the conversion product which contained methyl acrylate in 81.3% molar conversion, 81.0% molar yield, and 99.7% molar selectivity. No methyl propionate was detected.

### Example 5

Nitrogen (7 mL/min) and a liquid feed consisting of 40 wt. % acetic acid in MAPA (0.094 mL/min) were flowed over 10 g fused quartz (silicon oxide ground and sieved to 300-500 μm) at 560±10° C. and atmospheric pressure to give a GHSV of 225 h⁻¹ and a MAPA gas concentration of 30 mol. %. The reaction was equilibrated for 1 hour under flow then sampled every hour for 3 hours. The total methyl acrylate was determined by GC and HPLC analyses of three 1 hour pulls and results were averaged to give the conversion product which contained methyl acrylate in 98.9% molar conversion, 98.1% molar yield, and 99.2% molar selectivity; and methyl propionate in a molar yield of 0.4%.

### Example 6

### Comparative

Nitrogen (23 mL/min) and a liquid feed consisting of 40 wt. % acetic acid in MAPA (0.032 mL/min) were flowed over 10 g fused quartz (silicon oxide ground and sieved to 300-500 μm) at 560±10° C. and atmospheric pressure to give a GHSV of 225 h⁻¹ and a MAPA gas concentration of 10 mol. %. The reaction was equilibrated for 1 hour under flow then sampled every hour for 3 hours. The total methyl acrylate was determined by GC and HPLC analyses of three 1 hour pulls and results were averaged to give the conversion product which contained methyl acrylate in 99.8% molar conversion, 56.4% molar yield, and 56.5% molar selectivity; and methyl propionate in a molar yield of 3.5%.

### Example 7

### Comparative

Nitrogen (22 mL/min) and a liquid feed consisting of 2 wt. % acetic (0.054 mL/min) were flowed over 10 g fused quartz (silicon oxide ground and sieved to 300-500 μm) at 560±10° C. and atmospheric pressure to give a GHSV of 225 h⁻¹ and a MAPA gas concentration of 30 mol. %. The reaction was equilibrated for 1 hour under flow then sampled every hour for 3 hours. The total methyl acrylate was determined by GC and HPLC analyses of three 1 hour pulls and results were averaged to give the conversion product which contained the methyl acrylate in 99.2% molar conversion, 71.1% molar yield, 71.7% molar selectivity; and methyl propionate in a molar yield of 3.1%.

Table 1, below summarizes the data obtained from experiments described in the Examples 2 through 7, wherein “ND” means not detected:

TABLE 1

| | Acetic acid | MAPA | | | Methyl | | |
|---|---|---|---|---|---|---|---|
| | concentration | concentration | | MAPA | acrylate | Molar yield | |
| | in liquid | in gaseous | GHS | molar | molar | Methyl | Methyl |
| | feed | mixture | V | conversion | selectivity | acrylate | propionate |
| Example | wt. % | mol. % | h⁻¹ | % | % | % | % |
| 2 | 2 | 10 | 225 | 98.7 | 92.7 | 91.5 | 0.4 |
| 3 | 20 | 20 | 400 | 98.5 | 100 | 98.7 | ND |
| 4 | 2 | 10 | 1800 | 81.3 | 99.7 | 81.0 | ND |
| 5 | 40 | 30 | 225 | 98.9 | 99.2 | 98.1 | 0.4 |
| 6 | 40 | 10 | 225 | 99.8 | 56.5 | 56.4 | 3.5 |
| 7 | 2 | 30 | 225 | 99.2 | 71.7 | 71.1 | 3.1 |

### Example 8

An experiment was performed to determine the suitability of synthetic diamonds a substitute for the fused quartz used in the preceding Examples. Nitrogen (80 mL/min) and a liquid feed consisting of 2 wt. % acetic acid in MAPA (0.12 mL/min) were flowed over 19.2 g synthetic diamonds (60-80 mesh) at 560±10° C. and atmospheric pressure to give a GHSV of 900 h⁻¹ and a MAPA gas concentration of 20.8 mol. %. The reaction was equilibrated for 1 hour under flow then sampled every hour for 3 hours. The total methyl acrylate was determined by GC and HPLC analysis of three 1 hour pulls and results were averaged to give the methyl acrylate product in 93.9% molar conversion, 91.4% molar yield, and 97.4% molar selectivity. No methyl propionate was detected.

### Example 9

This example demonstrates the manufacture of acrylic acid from methyl acrylate in vapor phase with water and no organic carboxylic acid in accordance with an embodiment of the invention. A 316 stainless steel tubular reactor with inner diameter of 10.9 mm was packed with 8.5 g of γ-Al₂O₃ (Puralox SCCa—5/200; Sasol; Germany; bed volume 9.52 cm³). The reactor was purged by flowing nitrogen gas (15 mL/min) and heating to 400° C. for 4 hours and atmospheric pressure to pre-treat the catalyst. The conditions were then maintained at 275° C., atmospheric pressure and 10 mL/min nitrogen gas during the course of the reaction. Methyl acrylate (Sigma Aldrich Co. LLC, catalog #M27301) in water (4 wt. %) was vaporized and fed into the catalytic bed at a flow rate of 2.28 mL/h together with 10 mL/min of nitrogen as a carrier gas. The liquid effluents of the reaction were collected in a catch tank and analyzed off-line by HPLC, while the gaseous components were analyzed on-line by GC for the three 1 hour pulls and averaging the results. Under the tested conditions, the molar yield and selectivity for acrylic acid were 87% and 99%, respectively.

### Example 10

The methyl acrylate solution obtained from Example 2 can be diluted with water to give a 4 wt. % methyl acrylate solution and can be thereafter used as the feed in a second 316 stainless steel tubular reactor with inner diameter of 10.9 mm packed with 8.5 g of γ-Al₂O₃ (Puralox SCCa—5/200, Sasol, bed volume 9.52 cm³). The reactor can be purged by flowing nitrogen gas (15 mL/min) and heating to 400° C. for 4 hours and atmospheric pressure to pre-treat the catalyst. Thereafter, the conditions can be maintained at 275° C., atmospheric pressure and 10 mL/min nitrogen gas during the course of the ensuing reaction. Methyl acrylate in water (4 wt. %) can be vaporized and fed into the catalytic bed at a flow rate of 2.28 mL/h together with 10 mL/min of nitrogen as a carrier gas. The liquid effluents of the reaction can be collected in a catch tank and isolated for further isolation and purification.

### Example 11

An aliquot of 1.52 mL of sodium hydroxide in water (10 N) was added to a suspension of 1.29 g of methyl acrylate in 2.12 g. of water contained in a glass vial and under rapid mixing. The suspension was incubated at room temperature until a single phase was formed, followed by HPLC analysis and ¹H- and ¹³C-NMR spectroscopy characterization. Under the tested conditions, the molar yield of sodium acrylate was 95%.

### Example 12

To water (75 mL) was added sodium hydroxide (21.6 g, 0.541 mol). To this solution at ambient temperature was added methyl acrylate (50 mL, 46.6 g, 0.541 mol) resulting in an exotherm. The reaction was stirred at reflux for 30 minutes and cooled to room temperature. The resulting solution was stirred until complete by HPLC. To this solution was added ethyl acetate (50 mL). The ethyl acetate layer was separated and discarded to remove impurities. Concentrated 12 N HCl (45.1 mL, 0.541 mol) was dissolved in water (45 mL) and was added slowly to the reaction with water cooling to maintain temperature 20-25° C. The reaction was stirred for 1 hour then extracted twice with ethyl acetate (250 mL, 100 mL). The combined ethyl acetate layers were dried with sodium sulfate, filtered and washed with ethyl acetate. To the filtrate was added benzothiozine (0.05 g). The solution was evaporated at 30 mbar and 30° C. to give product as crude acrylic acid (30.9 g, 79.3% yield, 87.7% purity by ¹H-NMR). The crude acrylic acid was distilled at 70 mbar to give product as a clear colorless oil in three fractions. Fraction 1 gave 4.55 g acrylic acid (11.7% yield, 88.5% acrylic acid, 11.5% ethyl acetate by ¹H-NMR). Fraction 2 gave 2.17 g acrylic acid (5.6% yield, 97.6% acrylic acid, 2.4% ethyl acetate). Fraction 3 gave 18.63 g acrylic acid (47.8% yield, 98.9% acrylic acid, 0.4% ethyl acetate, 0.7% 3-hydroxypropanoic acid).

### Example 13

Methyl acrylate can be prepared as in Examples 2 to 7. To the resulting solution can be added a solution of sodium hydroxide in water. The reaction can be stirred for 30 minutes at 80° C. and cooled to room temperature. The resulting solution can be stirred until complete by HPLC. To this solution can be added ethyl acetate. Thereafter, the ethyl acetate layer can be separated and discarded to remove impurities. Concentrated 12 N HCl can be dissolved in water and can be added slowly to the reaction with water cooling to maintain the temperature at about 20 to about 25° C. The reactants can be stirred for 1 hour then extracted twice with ethyl acetate. The combined ethyl acetate layers can be dried with sodium sulfate, filtered and washed with ethyl acetate. To the filtrate can be added benzothiozine as a polymerization inhibitor. The solution can be evaporated at 30 mbar and 30° C. to give product as a crude acrylic acid mixture.

The foregoing description is given for clearness of understanding only, and no unnecessary limitations should be understood therefrom, as modifications within the scope of the invention may be apparent to those having ordinary skill in the art.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method comprising contacting (a) a gaseous mixture comprising (i) an inert gas and (ii) a vaporized liquid feed comprising methyl-2-acetoxy propionate (MAPA) and an excipient, with (b) a material having a surface acidity of about 5 micromoles per gram (μmol/g) or less and a surface basicity of about 15 μmol/g or less, under conditions sufficient to produce a conversion product comprising methyl acrylate in a molar yield of at least about 85% from MAPA, wherein the excipient is selected from the group consisting of acetic acid, formic acid, methyl acetate, lactic acid, carbon dioxide, and mixtures thereof.

2. The method of claim 1, wherein the excipient is acetic acid.

3. The method of claim 1, wherein the conditions comprise a gas hourly space velocity of about 225 per hour (h⁻¹) to about 900 h⁻¹.

4. The method of claim 2, wherein the excipient is present in the liquid feed in a concentration of about 20 wt. % to about 40 wt. %, based on the total weight of MAPA and excipient in the liquid feed; and, MAPA is present in the gaseous mixture in a concentration of about 20 mol. % to about 30 mol. %, based on the total moles of the gaseous mixture.

5. The method of claim 2, wherein the excipient is present in the liquid feed in a concentration of about 2 wt. % to about 20 wt. %, based on the total weight of MAPA and excipient in the liquid feed; and, MAPA is present in the gaseous mixture in a concentration of about 5 mol. % to about 20 mol. %, based on the total moles of the gaseous mixture.

6. The method of claim 1, wherein the inert gas is selected from the group consisting of nitrogen, helium, neon, argon, carbon monoxide, carbon dioxide, and mixtures thereof.

7. The method of claim 1, wherein the conditions comprise a temperature of about 500° C. to about 580° C.

8. The method of claim 1, wherein the molar yield of methyl acrylate from MAPA is at least about 95%.

9. The method of claim 1, wherein the conversion product comprises methyl propionate and the molar yield of methyl propionate from MAPA is about 5% or less.

10. The method of claim 1, wherein the material is selected from the group consisting of silicates, aluminates, carbon, titanium oxides, and mixtures thereof.

11. The method of claim 10, wherein the silicate is a fused quartz.

12. The method of claim 10, wherein the carbon is diamond.

13. The method of claim 1 further comprising contacting the conversion product in a vaporized mixture comprising at least one of water and an organic carboxylic acid with a catalyst under conditions sufficient to produce a reaction product comprising acrylic acid in a molar yield of at least about 80% from methyl acrylate.

14. The method of claim 13, wherein the organic carboxylic acid is selected from the group consisting of formic acid, acetic acid, butyric acid, isobutyric acid, pentanoic acid, 3-methylbutanoic acid, and mixtures thereof.

15. The method of claim 13, wherein the vaporized mixture further comprises an inert gas.

16. The method of claim 15, wherein the inert gas is selected from the group consisting of nitrogen, argon, helium, carbon monoxide, carbon dioxide, and mixtures thereof.

17. The method of claim 13, wherein the vaporized mixture comprises a molar ratio of (a) methyl acrylate to (b) the at least one of water and organic carboxylic acid of about 1:80 to about 1:200.

18. The method of claim 13, wherein the catalyst is selected from the group consisting of alumina, silica, silicates, aluminosilicates, silicoaluminophosphates, aluminophosphates, metal oxides, Group I and II sulfates, phosphates, pyrophosphates, polyphosphates, polysulfonate salts, and mixtures thereof.

19. The method of claim 18, wherein the catalyst comprises γ-alumina.

20. The method of claim 13, wherein the catalyst has a surface area of at least about 1 m²/g.

21. The method of claim 13, wherein the catalyst has a surface acidity of at least about 300 μmol/g.

22. The method of claim 13, wherein the conditions sufficient to produce the reaction product comprise a reaction temperature of about 150° C. to about 500° C.

23. The method of claim 13, wherein the conditions sufficient to produce the reaction product comprise a GHSV of about 180 h⁻¹ to about 1800 h⁻¹.

24. The method of claim 13, wherein the reaction product comprises propanoic acid, and the molar yield of propanoic acid from methyl acrylate is about 5% or less.

25. The method of claim 1 further comprising combining the conversion product with an aqueous solution of a base under conditions sufficient to produce a reaction product comprising a salt of acrylic acid in a molar yield of at least about 80% from methyl acrylate.

26. The method of claim 25, wherein the base is selected from at least one of hydroxides, carbonates, and bicarbonates of: ammonium, Group I metals, or Group II metals.

27. The method of claim 26, wherein the base is an alkali hydroxide selected from the group consisting of lithium hydroxide, sodium hydroxide, potassium hydroxide, rubidium hydroxide, cesium hydroxide, and mixtures thereof.

28. The method of claim 25, wherein the conditions sufficient to produce the reaction product comprise a temperature of 0° C. to about 100° C.

29. The method of claim 1, wherein the inert gas is nitrogen, the excipient is acetic acid, the material having a surface acidity of about 5 micromoles per gram (μmol/g) or less and a surface basicity of about 15 μmol/g or less is fused quartz, the conditions sufficient to produce a conversion product comprising methyl acrylate in a molar yield of at least about 90% from MAPA include a temperature of about 560° C., atmospheric pressure, and a GHSV of about 450 h⁻¹ and wherein MAPA is present in the gaseous mixture in a concentration of about 20 mol. %, based on the total moles of the gaseous mixture, and acetic acid is present in the liquid feed in a concentration of about 20 wt. %, based on the total weight of MAPA and excipient in the liquid feed.

30. The method of claim 1, wherein the inert gas is nitrogen, the excipient is acetic acid, the material having a surface acidity of about 5 micromoles per gram (μmol/g) or less and a surface basicity of about 15 μmol/g or less is fused quartz, the conditions sufficient to produce a conversion product comprising methyl acrylate in a molar yield of at least about 90% from MAPA include a temperature of about 560° C., atmospheric pressure, and a GHSV of about 225 h⁻¹ and wherein MAPA is present in the gaseous mixture in a concentration of about 30 mol. %, based on the total moles of the gaseous mixture, and acetic acid is present in the liquid feed in a concentration of about 40 wt. %, based on the total weight of MAPA and excipient in the liquid feed.
